(19) United States
(12) Patent Application Publication
(10) Pub. No.: US 2018/0265112 A1
(43) Pub. Date: Sep. 20, 2018

(54) MEDICAL DEVICE

(71) Applicant: General Electric Company, Schenectady, NY (US)

(72) Inventors: Yalan Yang, WuXi (CN); Zibo Yang, WuXi (CN); Qingqiang Li, WuXi (CN)

(21) Appl. No.: 15/920,398

(22) Filed: Mar. 13, 2018

(30) Foreign Application Priority Data
Mar. 20, 2017 (CN) 201710165289.6

(51) Int. Cl. B62B 3/00; A61B 6/00; A61B 8/00; B60B 33/04; B60B 33/00

(52) U.S. Cl. CPC B62B 3/008; B62B 2202/56; A61B 8/4405; B60B 33/045; B60B 33/0002; B60B 33/0042; B60B 33/0057; F16F 2224/02; F16F 2224/025; F16F 2224/0225; B62B 2202/00; B62B 2301/044; B62B 2301/05; B62B 2301/20; A61B 6/4405

(57) ABSTRACT

The present invention provides a medical device comprising: a main body; and two or more fixed casters and at least one floating caster, which are connected to a bottom portion of said main body and support said main body, wherein said floating caster is capable of floating in the vertical direction relative to said main body.

### FIELD OF THE INVENTION

The present invention relates to a medical device, and in particular to a movable medical device.

### BACKGROUND

Movable medical devices are becoming increasingly common. In medical devices, such as movable X-ray devices and movable ultrasonic diagnostic devices, casters are often used to support a main body of the medical device. In the prior art, multiple (e.g., four) fixed casters are generally arranged below the main body of such a movable medical device to support and move the medical device.

However, due to the tolerances in the manufacturing processes of the main body of the medical device and the casters themselves, it is possible that the multiple fixed casters are not in the same plane, making it unable to support the main body of the medical device on the fixed casters.

Therefore, it is desirable to provide a medical device in which multiple casters can be located in the same plane so as to provide stable support.

### BRIEF DESCRIPTION OF THE INVENTION

An exemplary embodiment of the present invention provides a medical device comprising: a main body; and two or more fixed casters and at least one floating caster, which are connected to a bottom portion of said main body and support said main body, wherein said floating caster is capable of floating in the vertical direction relative to said main body.

The medical device of the present invention uses a floating caster which is capable of floating relative to the main body so that the casters can be easily located in the same plane, thereby reducing the requirements of the manufacturing processes of the main body and the casters.

Other features and aspects will become apparent from the following detailed description, the drawings, and the claims.

### BRIEF DESCRIPTION OF THE DRAWINGS

The present invention will be better understood by the description of the exemplary embodiments of the present invention in conjunction with the accompanying drawings. In the drawings:

FIG. 1 is a schematic view of a medical device provided in an embodiment of the present invention;

FIG. 2 is an exploded view showing the structure of a floating caster and relevant parts of a main body of the medical device in an embodiment of the present invention;

FIGS. 3 and 4 are schematic views showing the structure of the floating caster, the relevant parts of the main body and the floating state of the floating caster of the medical device in the embodiment of the present invention; and

FIG. 5 is a schematic view showing an example of a medical system using the medical device in the embodiment of the present invention.

### DETAILED DESCRIPTION OF THE INVENTION

The specific embodiments of the present invention will be described below, and it should be noted that, in the specific description of these embodiments, for the sake of clarity, the specification may not describe all the details of each embodiment. It should be understood that, in the actual implementation of any embodiment, just as in the course of any engineering project or design project, in order to achieve the developers' specific goals and in order to meet system-related or business-related restrictions, a variety of design choices will be made, which may change based on the specific embodiment. Furthermore, it should be understood that, although the efforts made in this development process may be complicated and lengthy, but for those skilled in the art related to the content disclosed in the present invention, some changes in the design, manufacture or production on the basis of the technical content disclosed in this disclosure are merely conventional technical means, and it should not be construed as the content of the present disclosure being insufficient.

Unless otherwise defined, technical or scientific terms used in the claims and specification should have the general meaning understood by those skilled in the art related to the present invention. The terms “first”, “second” and the like used in the specification and the claims in the present invention are merely used to distinguish different components, rather than denoting any order, quantity, or importance. The term “one” or “a” or the like denotes the presence of at least one, rather than representing a quantitative restriction. The term “comprising” or “including” or the like means that the element or object before the term “comprising” or “including” encompasses the element or object or equivalent element listed after the term “comprising” or “including” while not excluding other elements or objects. The term “connected” or “connected to each other” or the like is neither limited to physical or mechanical connections, nor to direct or indirect connections.

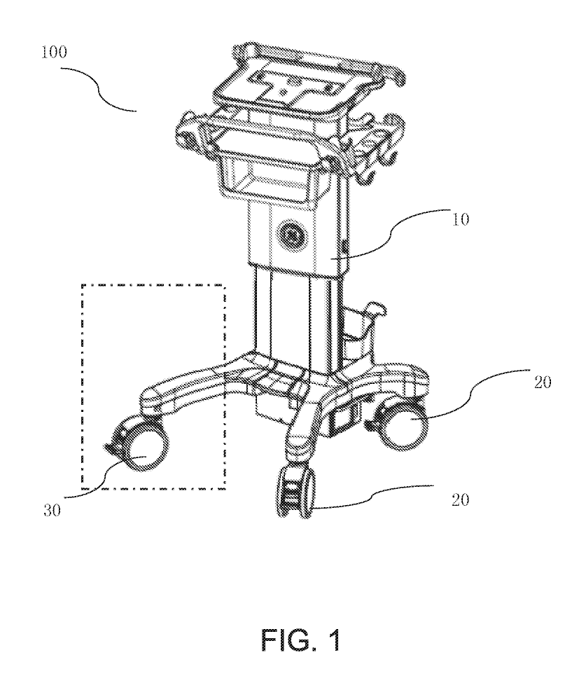

According to an embodiment of the present invention, a medical device is provided. FIG. 1 is a schematic view of the medical device in this embodiment. As shown in FIG. 1, the medical device 100 comprises: a main body 10, and two or more fixed casters 20 and at least one floating caster 30, which are connected to a bottom portion of the main body 10 and support the main body 10, wherein the floating caster 30 is capable of floating in the vertical direction relative to the main body 10.

In one example, the floating caster 30 is capable of elastically floating in the vertical direction relative to the main body 10.

As an example, the medical device shown in FIG. 1 takes the form of a cart for an ultrasonic diagnostic apparatus, on which a main frame of the ultrasonic diagnostic apparatus (not shown in FIG. 1) may be placed. However, it would be understood by those skilled in the art that the medical device of this embodiment may be any other suitable medical device, such as a movable X-ray apparatus or a movable medical bed. For convenience of illustration, the medical device of this embodiment is still described hereafter with a cart for an ultrasonic diagnostic apparatus as an example. However, it will be understood by those skilled in the art that these descriptions are similarly applicable to movable medical devices in other forms.

As an example, in the medical device of FIG. 1, the number of fixed casters 20 is three (one of which is not visible in this view), the number of floating casters 30 is one, and the fixed casters and the floating caster support the main body 10 at the four corners of the main body 10. This configuration is particularly advantageous for supporting the main body with all the casters being coplanar. As three points define a plane, the three fixed casters will be located in the same plane even if the main body and the fixed casters have certain manufacturing tolerances. The range of elastic floating of the floating caster may be set according to the manufacturing tolerances of the main body and the fixed casters, such that when the medical device is placed on a substantially flat floor, the floating caster can be adaptively coplanar with the three fixed casters via elastic floating.

Alternatively, a configuration of, for example, two fixed casters and one or two floating casters may also be used. The configuration using at least one floating caster is further beneficial to provide, in addition to the coplanar support, a buffer when moving on an uneven floor so as to reduce the impact on the main body of medical device.

In one example, the floating caster itself may have the same basic structure as that of the fixed caster, i.e., consisting of an axle, a wheel capable of rotating about the axle, and a bracket fixed to the axle (the bracket being used for connecting the caster to the main body supported by the caster). The difference between the floating caster and the fixed caster may lie in that the floating caster is connected to the main body in a different way, i.e., the floating caster is connected below the main body in such a way that the floating caster is capable of elastically floating in the vertical direction with respect to the main body.

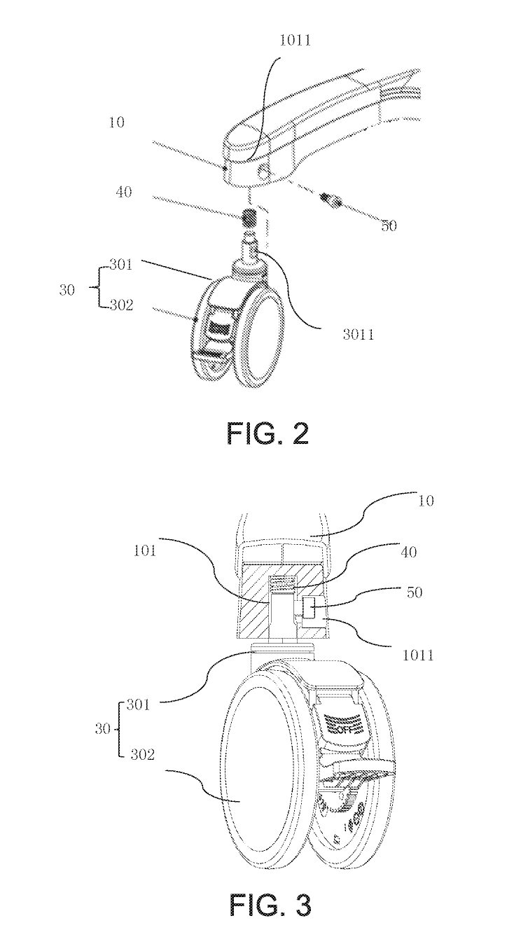

FIGS. 2 to 4 show a schematic representation of a floating castor in accordance with an embodiment. The floating castor, which is connected below the main body in such a way that the floating caster is capable of elastically floating in the vertical direction with respect to the main body, and the corresponding portion of the main body (i.e., the portion shown in dotted line in FIG. 1).

As an example, the floating caster 30 in FIGS. 2 to 4 has a two-wheel structure, comprising an axle (not shown), a bracket 301 fixedly connected to the axle, and two wheels 302 rotatably connected to the axle. Any other structure, such as a single-wheel caster, may be used as long as it can be connected to the main body in the elastic floating manner as described below.

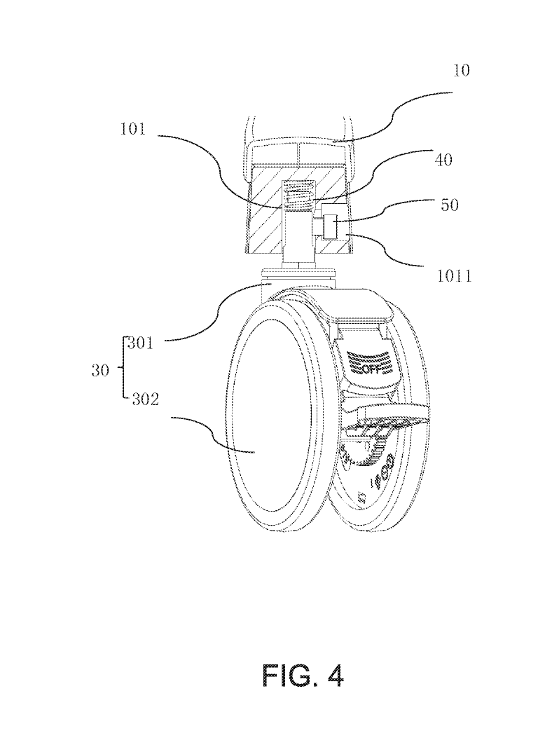

As shown in FIGS. 2 to 4, the floating caster 30 comprises a bracket 301 for connecting the floating caster 30 and the main body 10. A groove 101 accommodating one end of the bracket 301 is provided below the main body 10 at a position corresponding to the floating caster 30 (see FIG. 3 and FIG. 4; not shown in FIG. 2). An elastic member 40 is arranged in the groove 101. The lower end of the elastic member 40 is connected to the bracket 301, and the upper end of the elastic member 40 is connected to the top portion of the groove 101. It should be noted that the “connection” between the elastic member and one end of the bracket and the groove is not limited to a fixed connection, but may indicate the contact or abutment between the elastic member and the two latter components. The elastic member 40 may be preloaded to provide an elastic force that drives the floating caster 30 to elastically float.

In this example, the inner diameter of the groove 101 is slightly greater than the outer diameter of one end of the bracket 301, such that when one end of the bracket 301 is accommodated in the groove 101, the bracket 301 and the groove 101 may move relative to each other in the vertical direction, but substantially do not move relative to each other in the horizontal direction.

Optionally, the elastic member 40 may be a spring. Alternatively, the elastic member 40 may be made of an elastic material, such as a resin, rubber or sponge. The elastic member 40 may have various shapes, such as a dish shape, a column shape, or a cylindrical shape.

Optionally, as shown in FIGS. 2 to 4, in this example, the elastic member 40 has a hollow cylindrical shape (which is shown as a spring in the figures, but may also be a hollow cylinder made of another elastic material). Said one end of the bracket 301 has a stepped shape with the radial dimension of the lower part being greater than the radial dimension of the upper part. A top surface of the lower part of said one end of the bracket 301 abuts against the lower end of the elastic member 40, and the upper part is inserted into the elastic member 40.

In one example, said one end of the bracket 301 and the groove 101 may be each provided with a limit member for defining the range of relative movement between the floating caster 30 and the main body 10.

In one example, as shown in FIGS. 3 to 4, the limit member may comprise a limit rod 50 extending from said one end of the bracket 301 to the side wall of the groove 101 and a limit hole 1011 provided along the side wall of the groove 101. The limit rod 50 extends into the limit hole 1011 and is able to move up and down in the limit hole 1011.

Optionally, as shown in FIGS. 2 to 4, the limit rod 50 may be a limit bolt. Said one end of the bracket 301 is provided with a threaded hole 3011 which is fitted with the limit bolt 50, and the limit bolt 50 can be fastened in the threaded hole 3011.

The range in which the limit rod 50 moves up and down in the limit hole 1011 defines the maximum amount of elastic deformation of the elastic member, that is, defines the range in which the floating caster elastically floats in the vertical direction with respect to the main body. For example, the dimension of the limit hole 1011 may be designed according to the manufacturing tolerances of the main body and the casters of the medical device, so that the maximum range in which the limit rod 50 can move up and down in the limit hole 1011 is, for example, 5 mm.

When the floating caster is in a position higher than the plane where the fixed casters are located, due to the manufacturing tolerances of the main body and the casters or the unevenness of the floor, the preloaded elastic member 40 is further compressed to decrease the height of the main body under the support of the floating caster, so that the floating caster may be coplanar with the fixed casters or may be equivalent to a coplanar support for the main body. FIG. 3 shows a state in which the limit rod 50 of the floating caster is in the uppermost position of the limit hole 1011 (i.e. at the minimum height under the support of the floating caster), and at this moment the elastic member 40 in the form of a spring is compressed to a height equal to the stepped-shaped upper part of one end of the bracket 301.

On the other hand, when the floating caster is in a position lower than the plane where the fixed casters are located, due to the manufacturing tolerances of the main body and the casters or the unevenness of the floor, the amount of compression of the elastic member 40 becomes smaller, and the restoring elastic force of the elastic member pushes down the caster to increase the height of the main body under the support of the floating caster, so that the floating caster may be coplanar with the fixed casters or may be equivalent to a coplanar support for the main body. FIG. 4 shows a state in which the limit rod 50 is in the lowermost position of the limit hole 1011 (i.e. at the maximum height under the support of the floating caster), and at this moment the elastic member 40 in the form of a spring is restored to be higher than the stepped-shaped upper part of one end of the bracket 301.

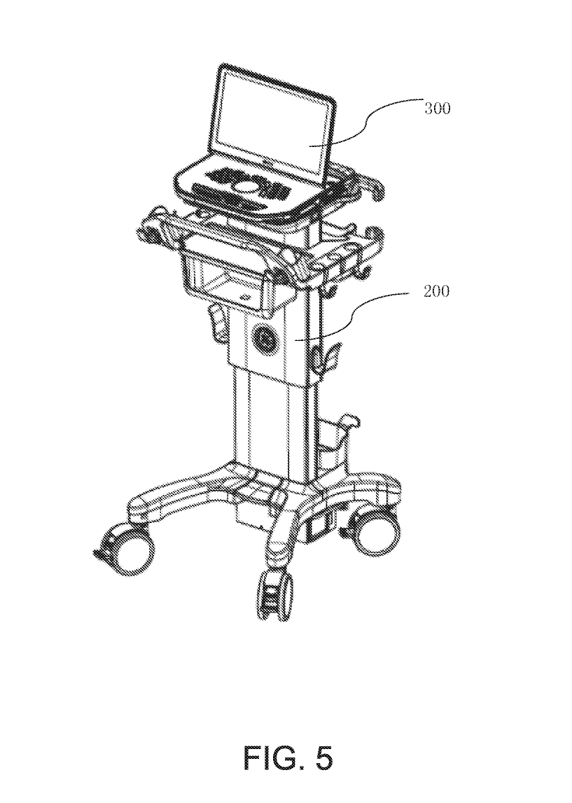

The medical device of this embodiment can be applied to various medical systems. As described above, in one example, the medical device may be a cart for an ultrasonic diagnostic apparatus, and may be used in an ultrasound diagnostic system. FIG. 5 shows an ultrasonic medical system using the cart for an ultrasonic diagnostic apparatus in this example. The system comprises a medical device 200 in the form of a cart, and an ultrasonic main frame 300 disposed on the medical device 200. The medical device 200 has a structure similar to the medical device 100 described above with reference to FIGS. 1 to 4, and will not be described here again.

Some exemplary embodiments have been described above. However, it should be understood that various modifications can be made. For example, if the techniques as described are performed in a different order and/or if the components in the system, architecture, device or circuit as described are combined in different ways and/or are replaced or supplemented with a further component or an equivalent thereof, an appropriate result can be achieved. Accordingly, other embodiments also fall within the scope for protection of the claims.

We claim:

1. A medical device, comprising:
a main body; and
two or more fixed casters and at least one floating caster, which are connected to a bottom portion of said main body and support said main body, wherein said floating caster is capable of floating in the vertical direction relative to said main body.

2. The medical device of claim 1, wherein said floating caster is capable of elastically floating in the vertical direction relative to the main body.

3. The medical device of claim 1, wherein said floating caster comprises a bracket for connecting said floating caster and said main body, a groove for accommodating one end of said bracket is provided below said main body at a position corresponding to said floating caster, an elastic member is arranged in said groove, a lower end of said elastic member is connected to said one end of said bracket, and an upper end of said elastic member is connected to a top portion of said groove.

4. The medical device of claim 3, wherein said elastic member has a hollow cylindrical shape, and said one end of said bracket has a stepped shape with the radial dimension of the lower part being greater than the radial dimension of the upper part, a top surface of the lower part of said one end of said bracket abuts against the lower end of said elastic member, and the upper part of said one end of said bracket is inserted into the elastic member.

5. The medical device of claim 3, wherein said one end of said bracket and said groove are each provided with a limit member which defines the range of relative movement between said floating caster and said main body.

6. The medical device of claim 5, wherein said limit member comprises a limit rod extending from said one end of said bracket to a side wall of said groove and a limit hole provided along the side wall of said groove, and said limit rod extends into said limit hole and is able to move up and down in said limit hole.

7. The medical device according to claim 6, wherein said limit rod is a limit bolt, said one end of said bracket is provided with a threaded hole which is fitted with said limit bolt, one end of said limit bolt is fastened in said threaded hole and the other end of said limit bolt extends into said limit hole.

8. The medical device of claim 3, wherein said elastic member is a spring.

9. The medical device of claim 3, wherein said elastic member is made of a resin, rubber, or sponge.

10. The medical device according to claim 1, wherein the number of said fixed casters is three, the number of said floating casters is one, and said fixed casters and said floating caster respectively support said main body at four corners of said main body.
